(19) United States
(12) Patent Application Publication
(10) Pub. No.: US 2022/0211500 A1
(43) Pub. Date: Jul. 7, 2022

(54) TISSUE REMODELING SYSTEM AND METHODS

(71) Applicant: Heartchord Medical, Inc., Petaluma, CA (US)

(72) Inventor: Jan R. Lau, Windsor, CA (US)

(21) Appl. No.: 17/567,508

(22) Filed: Jan. 3, 2022

Related U.S. Application Data
(60) Provisional application No. 63/133,970, filed on Jan. 5, 2021.
(60) Provisional application No. 63/203,807, filed on Jul. 30, 2021.

(51) Int. Cl. A61F 2/24

(52) U.S. Cl. CPC A61F 2/2454; A61F 2/2466; A61F 2220/0075; A61F 2230/0091; A61F 2220/0016

(57) ABSTRACT

Systems and methods for remodeling of tissue, such as heart tissue. In some configurations, the systems and methods are directed toward remodeling of the mitral valve of a patient. The systems and methods can include a first tissue anchor, a second tissue anchor, a suture that extends between the tissue anchors, and a suture lock that secures the suture relative to at least one of the tissue anchors or fixes a length of the suture. The systems and methods can also include a suture trimmer that can trim an excess portion of the suture. In some configurations, the suture lock is reversible to allow for iterative adjustments of the remodeling. In some configurations, the tissue anchors include a feature or features, such as one or more barbs, to inhibit removal of the tissue anchor from an implanted position within tissue.

### BACKGROUND

### Field

The present disclosure relates to systems and methods for remodeling tissue. In particular, the present disclosure relates to systems and methods for heart valve remodeling, such as mitral valve remodeling.

### Description of Related Art

Heart valves lie at the exit of each of the four heart chambers. Heart valves work as one-way valves to prevent blood from flowing in the wrong direction. Each valve has a set of flaps, called leaflets or cusps. Valve regurgitation is when blood leaks through an incompletely closed valve, allowing blood flow in two directions during contraction. Regurgitation may be caused either due to an abnormality of the leaflets themselves (called primary regurgitation), such as valve prolapse, damaged chordae, rheumatic fever, endocarditis, trauma or congenital heart defects. On the other hand, in secondary regurgitation, the valve itself is intact and only the surrounding structures the valve leaflets insert into are abnormal, resulting in regurgitation. Examples for secondary regurgitation are history of heart attack, cardiomyopathy, prolong use of certain drugs, radiation, atrial fibrillation, etc. Regurgitation can result in congestive heart failure, which is the most common hospital admission diagnosis in the United States. Symptoms of congestive heart failure include fatigue, shortness of breath, swelling of feet and legs. Valve regurgitation leads to a vicious cycle of heart failure, arrhythmias, and worsening cardiomyopathy (weakening of the heart muscle), which results in more regurgitation.

Historically, open surgical valve repair or replacement is performed to treat diseases such as regurgitation. More recently, catheter-based technologies have been developed and introduced into clinical practice for the repair of the mitral valve. In general, repair is deemed superior to valve replacement to restore coaptation of the leaflets.

### SUMMARY

The systems, methods and devices described herein have innovative aspects, no single one of which is indispensable or solely responsible for their desirable attributes. Without limiting the scope of the claims, some of the advantageous features will now be summarized.

An aspect of the present disclosure involves a system for mitral valve remodeling that includes a first tissue anchor and a second tissue anchor. The first tissue anchor is configured to be implanted into tissue at a first location at or near an annulus of a mitral valve of a patient. The first tissue anchor comprises an anchor portion, a drive portion and a suture mount portion. The anchor portion engages the tissue and is implanted by rotation about a longitudinal axis of the first tissue anchor. The drive portion is rotatably fixed with respect to the anchor portion and is configured to removably engage with a drive member of a catheter. The suture mount portion is rotatable relative to the anchor portion and the drive portion and is located between the drive portion and the anchor portion along the longitudinal axis. The second tissue anchor is configured to be implanted into tissue at a second location at or near the annulus of the mitral valve across from the first location. The second tissue anchor comprises an anchor portion, a drive portion and a suture mount portion. The anchor portion engages the tissue and is implanted by rotation about a longitudinal axis of the second tissue anchor. The drive portion is rotatably fixed with respect to the anchor portion and is configured to removably engage with the drive member of the catheter. The suture mount portion is rotatable relative to the anchor portion and the drive portion and is located between the drive portion and the anchor portion along the longitudinal axis. A suture has a tensioned portion that extends between a first suture mount location on the suture mount portion of the first tissue anchor and a second suture mount location on the suture mount portion of the second tissue anchor. The suture mount portions of the first and second tissue anchors rotate to align with one another in response to tension applied to the suture.

In an embodiment, each of the first and second suture mount locations of the first and second tissue anchors comprises a passage that accommodates the suture, wherein the tensioned portion of the suture extends from an end of the passage relatively closer to the anchor portion.

In an embodiment, a suture lock is configured to secure a portion of the suture relative to the second tissue anchor to fix a length of a tensioned portion of the suture between the first tissue anchor and the second tissue anchor.

In an embodiment, the suture lock comprises a first portion and a second portion movable relative to the first portion, wherein a lock portion of the suture is captured between the first portion and the second portion.

In an embodiment, the first portion comprises a passage, wherein the lock portion of the suture passes through the passage.

In an embodiment, the first portion of the suture lock is configured to contact the suture mount portion of the second tissue anchor to fix the length of the tensioned portion of the suture.

In an embodiment, the passage of the suture lock is aligned with a passage of the second suture mount location of the second tissue anchor when the suture lock is in contact with the second tissue anchor.

In an embodiment, the first portion of the suture lock is rotationally fixed relative to the second portion.

In an embodiment, a threaded fastener is configured to move the first portion of the suture lock relative to the second portion.

In an embodiment, the threaded fastener is configured to move the first portion of the suture lock toward and away from the second portion.

In an embodiment, a suture cutter is configured to cut the suture.

In an embodiment, the suture cutter comprises a tip having an axial slot and a radial passage, wherein the axial slot intersects the radial passage, wherein the suture passes through the radial passage, the suture cutter further comprising a blade that is movable within the slot to cut the suture.

In an embodiment, the anchor portion comprises one or more barbs.

In an embodiment, each of the barbs comprises a tubular element having an angled end with a tip of the angled end located radially outward.

An aspect of the present disclosure involves a system for implanting a tissue anchor in heart tissue of a patient. The system includes a delivery catheter comprising an anchor delivery tip. The tip comprises a stationary portion and a rotatable portion. The stationary portion comprises a suture passage having a first end and a second end. The rotatable portion comprises a drive portion. The system further includes a tissue anchor comprising an anchor portion, a drive portion and a suture mount portion. The anchor portion engages the heart tissue and is implanted by rotation about a longitudinal axis of the tissue anchor. The drive portion is rotatably fixed with respect to the anchor portion and is configured to removably engage with the drive portion of the catheter. The suture mount portion is rotatable relative to the anchor portion and the drive portion. A suture is secured to the suture mount portion. The tissue anchor is configured to be engaged with the delivery catheter with the drive portion of the tissue anchor engaged with the drive portion of the delivery catheter. The suture extends through the suture passage of the tip of the delivery catheter such that the suture can be tensioned to restrain the suture mount portion of the tissue anchor from rotating as the rotatable portion of the tip of the delivery catheter is rotated to rotate the drive portion and the anchor portion of the tissue anchor to thereby implant the tissue anchor into the heart tissue.

In an embodiment, the suture passage of the stationary portion is located radially outward of the rotatable portion.

In an embodiment, the suture mount portion is located between the drive portion and the anchor portion.

In an embodiment, the delivery catheter comprises a distal tip cover configured to surround the tissue anchor prior to deployment.

In an embodiment, the distal tip cover comprises a slot through which the suture passes from exterior the distal tip cover to interior the distal tip cover such that the suture can be secured to the suture mount portion.

In an embodiment, the distal tip cover comprises a slit that extends from the slot to a distal end of the distal tip cover, wherein the slit is configured such that the suture can move from the slot, pass through the slit, and be separated from the distal tip cover when the tissue anchor is deployed from the delivery catheter.

In an embodiment, the anchor portion comprises one or more barbs.

In an embodiment, each of the barbs comprises a tubular element having an angled end with a tip of the angled end located radially outward.

An aspect of the present disclosure involves a tissue anchor including an anchor portion comprising a helical thread configured to be implanted into bodily tissue by rotation about a longitudinal axis of the tissue anchor. The tissue anchor further includes a drive portion that is rotatably fixed with respect to the anchor portion. The drive portion is configured to removably engage with a drive member of a catheter such that rotation of the drive member rotates the drive portion and the anchor portion of the tissue anchor. The tissue anchor further includes a suture mount portion is rotatable relative to the anchor portion and the drive portion. The suture mount portion is configured to connect to a suture at a suture mount location. The suture mount portion is configured to rotate to align the suture mount location with a direction of force of the suture. The suture mount portion is located between the drive portion and the anchor portion along the longitudinal axis.

In an embodiment, the helical thread of the anchor portion is a helical coil defining a hollow interior space.

In an embodiment, the helical coil comprises a circular cross-sectional shape.

In an embodiment, the drive portion defines a radially outward-facing drive surface that is configured to engage the drive member of the catheter.

In an embodiment, the drive portion comprises a square cross-sectional shape that defines the radially outward-facing drive surface.

In an embodiment, the suture mount portion has a peripheral surface surrounding the longitudinal axis of the tissue anchor, the peripheral surface defining a geometric center of the suture mount portion, wherein an axis of rotation of the suture mount portion is spaced from the geometric center.

In an embodiment, the suture mount location is on an opposite side of the geometric center from the axis of rotation.

In an embodiment, the suture mount location comprises a passage extending through the suture mount portion in a direction substantially aligned with the longitudinal axis of the tissue anchor.

In an embodiment, a length of the anchor portion along the longitudinal axis is greater than a length of one or both of the drive portion and the suture mount portion.

In an embodiment, the length of the drive portion is greater than the length of the suture mount portion.

In an embodiment, the anchor portion comprises one or more barbs.

In an embodiment, each of the barbs comprises a tubular element having an angled end with a tip of the angled end located radially outward.

An aspect of the present disclosure involves a tissue anchor including an anchor portion comprising a helical thread configured to be implanted into bodily tissue by rotation about a longitudinal axis of the tissue anchor. The tissue anchor further includes a drive portion that is rotatably fixed with respect to the anchor portion. The drive portion is configured to removably engage with a drive member of a catheter such that rotation of the drive member rotates the drive portion and the anchor portion of the tissue anchor. The tissue anchor further includes a suture mount portion that is rotatable relative to the anchor portion and the drive portion. The suture mount portion is configured to connect to a suture at a suture mount location. The suture mount portion is configured to rotate to align the suture mount location with a direction of force of the suture. The suture mount portion is located above the anchor portion along the longitudinal axis. The suture mount portion has a first end surface and a second end surface opposite the first end surface. The second end surface is closer to the anchor portion than the first end surface along the longitudinal axis. The suture mount portion is configured such that suture extends from the tissue anchor at or below the second end surface.

In an embodiment, the suture mount portion is located immediately adjacent the anchor portion.

In an embodiment, the helical thread of the anchor portion is a helical coil defining a hollow interior space.

In an embodiment, the helical coil comprises a circular cross-sectional shape.

In an embodiment, the drive portion defines a radially outward-facing drive surface that is configured to engage the drive member of the catheter.

In an embodiment, the drive portion comprises a square cross-sectional shape that defines the radially outward-facing drive surface.

In an embodiment, the suture mount portion has a peripheral surface surrounding the longitudinal axis of the tissue anchor, the peripheral surface defining a geometric center of the suture mount portion, wherein an axis of rotation of the suture mount portion is spaced from the geometric center.

In an embodiment, the suture mount location is on an opposite side of the geometric center from the axis of rotation.

In an embodiment, the suture mount location comprises a passage extending through the suture mount portion from the first end surface to the second end surface in a direction substantially aligned with the longitudinal axis of the tissue anchor.

In an embodiment, a length of the anchor portion along the longitudinal axis is greater than a length of one or both of the drive portion and the suture mount portion.

In an embodiment, the length of the drive portion is greater than the length of the suture mount portion.

In an embodiment, the anchor portion comprises one or more barbs.

In an embodiment, each of the barbs comprises a tubular element having an angled end with a tip of the angled end located radially outward.

An aspect of the present disclosure involves a suture lock for a tissue remodeling system. The suture lock includes a first portion comprising a base flange and a hub extending in an axial direction from the base flange. The base flange comprises a suture passage configured to accommodate a suture of the tissue remodeling system. The suture lock also includes a second portion comprising an end wall and at least one side wall defining a space to slidably engage the hub of the first portion. The end wall and the at least one side wall are configured to prevent rotation of the first portion when the hub is positioned within the space. The second portion further comprising a clamping surface located adjacent an end of the suture passage of the base flange and configured to clamp a portion of the suture against the base flange to fix the suture relative to the suture lock. The second portion is movable toward and away from the first portion to selectively clamp and release the suture.

In an embodiment, the at least one sidewall comprises a first side wall and a second side wall, wherein the first and second side walls are parallel and spaced apart from one another to receive the hub therebetween.

In an embodiment, the first portion comprises a threaded cavity extending in the axial direction within the hub and the second portion comprises an opening within the end wall, the suture lock further comprising a threaded fastener that passes through the opening and threadably engages the threaded cavity, wherein the threaded fastener is configured to move the first portion toward the second portion in response to rotation in a first direction and to allow the first portion to move away from the second portion in response to rotation in a second direction.

An aspect of the present disclosure involves a method of remodeling a mitral valve. The method includes implanting, using at least one catheter, a first tissue anchor at a first location at or near an annulus of a mitral valve of a patient. The method further includes implanting, using the at least one catheter, a second tissue anchor at a second location at or near the annulus of the mitral valve of the patient across from the first location. The method also includes extending a suture between the first tissue anchor and the second tissue anchor and using the suture to move the first tissue anchor and the second tissue anchor toward one another. The method includes fixing a tension length of the suture between the first tissue anchor and the second tissue anchor using a suture lock that is lockable using the at least one catheter. The method further includes observing the function of the mitral valve and, if desired, unlocking the suture lock, increasing or decreasing the tension length of the suture, and relocking the suture lock.

In an embodiment, the method further comprises cutting an excess portion of the suture using a suture cutter.

An aspect of the present disclosure involves a method of tensioning a suture of a mitral valve remodeling system. The method includes slidably engaging a suture lock with a suture that has an end fixed to a first tissue anchor implanted at a first location at or near an annulus of the mitral valve and is slidably engaged with a second tissue anchor implanted at a second location at or near the annulus of the mitral valve. The method further includes sliding the suture lock along the suture toward the second tissue anchor using a catheter until the suture lock contacts the second tissue anchor. The method also includes applying a pulling force to the suture while holding the suture lock in contact with the second tissue anchor to tension a portion of the suture extending between the first tissue anchor and the second tissue anchor.

In an embodiment, the method further includes locking the suture lock on the suture to maintain the tension of the portion of the suture extending between the first tissue anchor and the second tissue anchor.

In an embodiment, the method further includes disengaging the catheter from the suture lock after the suture lock is locked on the suture.

An aspect of the present disclosure involves a tissue anchor having an anchor portion comprising a helical thread configured to be implanted into bodily tissue by rotation in a first direction about a longitudinal axis of the tissue anchor. One or more barbs are configured to permit rotation of the tissue anchor in the first direction and inhibit rotation of the tissue anchor in a second direction opposite the first direction. The tissue anchor also comprises a drive portion that is rotatably fixed with respect to the anchor portion. The drive portion is configured to removably engage with a drive member of a catheter such that rotation of the drive member rotates the drive portion and the anchor portion of the tissue anchor. The tissue anchor also comprises a suture mount portion configured to connect to a suture at a suture mount location.

In an embodiment, the suture mount portion is rotatable relative to the anchor portion and the drive portion.

In an embodiment, the suture mount portion is configured to rotate to align the suture mount location with a direction of force of the suture.

In an embodiment, the suture mount portion is located between the drive portion and the anchor portion along the longitudinal axis.

In an embodiment, the one or more barbs comprises a plurality of barbs spaced from one another along a length of the helical thread.

In an embodiment, each of the one or more barbs comprises an angled end having a tip that is located radially outward on the barb.

In an embodiment, the angled end has an angle between 30-60 degrees.

In an embodiment, each of the one or more barbs is or comprises a tubular element secured to the helical thread.

In an embodiment, the tubular element is straight.

In an embodiment, the tubular element defines an interior passage having a diameter that is larger than a diameter of the helical thread to allow the tubular element to be advanced along the helical thread during manufacture.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 is a perspective view of a mitral valve remodeling system implanted in a mitral valve of a patient.

FIG. 2 is a perspective view of a tissue anchor of the system of FIG. 1.

FIG. 2A is a side elevation view of the tissue anchor of FIG. 2.

FIG. 2B is a top plan view of a suture mount portion of the tissue anchor of FIG. 2.

FIG. 3 is perspective view of a suture lock of the system of FIG. 1.

FIG. 4 is a sectional view of the suture lock of FIG. 3.

FIG. 5 is a view of a guide catheter and delivery catheter for use in implanting the system of FIG. 1.

FIG. 6 is a perspective view of the delivery catheter of FIG. 5.

FIG. 7 is a perspective view of a first tissue anchor being implanted at a first location in the mitral valve of a patient.

FIG. 8 is a perspective view of a second tissue anchor being implanted at a second location in the mitral valve of the patient.

FIG. 8A is a partial sectional view of a tip of a delivery catheter for delivering the tissue anchors.

FIG. 9 is a perspective view of a suture lock being placed at the second location in the mitral valve of the patient.

FIG. 9A is a sectional view of a tip of a delivery catheter for delivering the suture lock.

FIG. 10 is a perspective view of an excess portion of the suture being trimmed.

FIG. 11 is a process flow of a method for implanting and, optionally, adjusting a mitral valve remodeling system.

FIG. 12A is a perspective view of a portion of an alternative delivery catheter having a distal tip cover in which the tissue anchor is stowed.

FIG. 12B illustrates the tissue anchor deployed from the distal tip cover with the suture extending through a slot in the distal tip.

FIG. 12C illustrates the suture passing through a slit in the distal tip cover.

FIG. 13 illustrates an alternative tissue anchor having a plurality of barbs on the anchor portion.

FIG. 14 illustrates a tissue anchor and suture lock configured to be secured to the tissue anchor separately from the suture.

FIG. 15 illustrates a remodeling system having a blocking element that is configured to retain a portion of the suture and the suture lock relative to the associated tissue anchor.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The percutaneous technology described in this application is designed to treat valve regurgitation by structurally changing the heart to increase leaflet coaptation. The technology may be applied to either atrio-ventricular valve of the heart (mitral and tricuspid valve). The concept of repair is an annular approach of valve repair.

There are several advantages of one or more embodiments of the technology described within this application compared to currently either commercially available or currently developed, experimental technology. Those advantages include one or more of the following:
1. In one or more embodiments, the disclosed technology allows individualization of regurgitation reduction, depending on the underlying pathology and valve size (specifically, where anchors are placed and how much the chord is tethered). From a practical point of view, the disclosed technology eliminates the need for hospitals to acquire a large range of devices of different sizes. Substantially the only equipment necessary is delivery catheters, anchors, and chord.
2. The disclosed technology conceptually may be particularly helpful in so far unstudied patient populations, such as those with secondary mitral regurgitation due to atrial pathologies or patients with tricuspid regurgitation due to pacemaker or defibrillator leads. Nevertheless, one or more embodiments of the disclosed technology may also prove effective in secondary mitral regurgitation due to ventricular disease, or even in select cases of primary mitral regurgitation.
3. In one or more embodiments, the disclosed technology may be used as an adjunct to existing technology (edge to edge repair) in cases where suboptimal results are present or anticipated.
4. Further advantages of one or more embodiments of the disclosed technology is its ability to permit other, future catheter-based valve repair or replacement strategies due to the ability to cut the repair chord.
5. As with most percutaneous repair strategies, one or more embodiments of the disclosed technology is anticipated to have a much shorter recovery time and better safety profile compared to open surgical repair or replacement.
6. Comparing the disclosed technology to other currently available or tested repair devices, the simplicity is striking. Procedure time and learning curve likely are favorable due to its simple design.
7. Finally, the smaller access of the delivery system of one or more embodiments of the disclosed technology likely will eliminate concerns about residual iatrogenic atrial septal defects following percutaneous, transseptal access for mitral valve repair and allows easy access via the right internal jugular vein for repair of the tricuspid valve.

The figures illustrate systems and methods for stabilizing or remodeling tissue. Preferably, the systems and methods disclosed are configured for remodeling soft tissue, such as heart tissue, for example. The illustrated systems and related methods are configured for remodeling the mitral valve. However, the system, components thereof and/or related methods could be used for other purposes or could be modified for use in other applications. For example, the disclosed systems, components or methods could be modified for use in stabilizing or remodeling other soft (e.g., muscle or connective tissue) or hard (e.g., bone) bodily tissues.

The illustrated systems are configured for percutaneous transvascular delivery using one or more catheters or other suitable conduits. However, in alternative arrangements or applications, the systems or components thereof as disclosed or as modified by one skilled in the art could be delivered to or installed at the desired bodily location by other means, such as by using a direct approach.

### System Overview

The illustrated system 100 for remodeling a mitral valve includes a first tissue anchor 102, a second tissue anchor 104, a suture 106 and a suture lock 110. The suture 106 extends between the first tissue anchor 102 and the second tissue anchor 104. The suture 106 can be secured relative to the first tissue anchor 102 and the second tissue anchor 104 to fix a distance between the tissue anchors 102, 104. The distance between the anchors 102, 104 can be adjusted to achieve a desired level of performance of the mitral valve. The suture lock 110 secures the suture 106 relative to the second tissue anchor 104 to maintain the desired distance between the anchors 102, 104.

The first tissue anchor 102 is implanted at a first location 112 in the heart tissue of a patient, which can be at or near the mitral valve 114. The second tissue anchor 104 is implanted at a second location 116, which can be at or near the mitral valve 114. Preferably, the first tissue anchor 102 and the second tissue anchor 104 are each implanted at or near the annulus 120 of the mitral valve 114. Preferably, each of the tissue anchors 102, 104 are located close enough to the annulus 120 so that the tissue has sufficient strength to support the tissue anchors 102, 104 without tearing or otherwise being compromised under normal or expected conditions.

In the illustrated arrangement, the first tissue anchor 102 and the second tissue anchor 104, or the first location 112 and the second location 116, are located on opposite sides of the mitral valve 114. In particular, the first tissue anchor 102 is located on the posterior leaflet 122 and the second tissue anchor 104 is located on the anterior leaflet 124. However, these positions could also be reversed. The first tissue anchor 102 can be located within a central region or at or near a midpoint of the posterior leaflet 122 in a direction along the sealing edge 126 of the mitral valve 114. The second tissue anchor 104 can be located within a central region or at or near a midpoint of the anterior/posterior leaflet 124 in a direction along the sealing edge 126 of the mitral valve 114.

The suture 106 has a first end 130 that is secured to the first tissue anchor 102. As used herein, the term suture can refer to any suitable line capable of connecting the tissue anchors 102, 104 and maintaining the tissue anchors 102, 104 at the adjusted separation distance (e.g., not stretching) under the expected conditions and for the expected life of the system 100, unless otherwise indicated. The suture 106 extends from the first tissue anchor 102 to the second tissue anchor 104. The suture 106 engages the second tissue anchor 104 such that the relative movement is permitted between the suture 106 and the second tissue anchor 104. In the illustrated configuration, the suture 106 slides within or relative to the second tissue anchor 104. A length of the suture 106 located between the tissue anchors 102, 104 can be adjusted to achieve a desired distance between the tissue anchors 102, 104. The distance between the tissue anchors 102, 104 can be adjusted to achieve a desired level of remodeling of the mitral valve 114 or a desired performance of the mitral valve 114.

The suture lock 110 can be secured at a desired location along a length of a portion of the suture 106 that is not located between the tissue anchors 102, 104. The suture lock 110 can contact the second tissue anchor 104 to limit a length of the suture 106 located between the tissue anchors 102, 104. When the suture 106 is used to remodel the mitral valve 114 by moving the first location 112 closer to the second location 116, the resiliency of the tissue of the mitral valve 114 will exert a force in a direction tending to move the anchors 102, 104 apart thereby tensioning the portion of the suture 106 located between the first tissue anchor 102 and the second tissue anchor 104. Accordingly, this portion of the suture 106 can be referred to herein as the tensioned length 132. Thus, in some configurations, the suture lock 110 is held against the second tissue anchor 104 by the tension of the tensioned length 132 of the suture 106. The suture lock 110 only fixes the maximum separation distance of the first tissue anchor 102 and the second tissue anchor 104, but permits the tissue anchors 102, 104 to move closer to one another.

In some configurations, as described further below, the suture lock 110 is reversible. That is, the suture lock 110 can be secured at a location along the length of the suture 106 to define a desired tensioned length 132. The performance of the mitral valve 114 can then be observed and, if desired, the suture lock 110 can be unsecured from the suture 106, moved to another location and once again secured to the suture 106 to define a different tensioned length 132. This process can be repeated until a desired level of remodeling or performance of the mitral valve 114 is obtained.

### Tissue Anchor

In some configurations, the tissue anchors 102, 104 are identical or substantially identical to one another. Accordingly, the first tissue anchor 102 is described. The second tissue anchor 104 can be identical or substantially identical, or can be of another suitable arrangement.

The illustrated tissue anchor 102 includes an anchor portion 140, a drive portion 142 and a suture mount portion 144 arranged along a longitudinal axis 148 of the tissue anchor 102. In some configurations, the suture mount portion 144 is located adjacent the anchor portion 140. In the illustrated configuration, the suture mount portion 144 is located between the anchor portion 140 and the drive portion 142 along the longitudinal axis 148.

The anchor portion 140 is configured to be implanted into tissue. Preferably, the anchor portion 140 is configured to be implanted into soft tissue, such as heart tissue. In some configurations, the anchor portion 140 is a threaded member that is implanted by rotation about the longitudinal axis 148. The illustrated anchor portion 140 comprises a helical member 150. The helical member 150 comprises an elongate member having a circular cross-section, which is wound about the longitudinal axis 148 to define an elongate hollow space 152 extending along the longitudinal axis 148. The anchor portion 140 defines a length 151 that is sufficient for the anchor portion 140 to be secured in the desired tissue.

The drive portion 142 is configured to be engaged by a catheter or other implantation tool to allow for implantation of the tissue anchor 102. The drive portion 142 is fixed for rotation with the anchor portion 140 such that rotation of the drive portion 142 results in rotation of the anchor portion 140.

The drive portion 142 includes a drive surface 154 configured to engage with a drive member of a catheter. In the illustrated arrangement, the drive surface 154 is non-circular in shape. In the illustrated arrangement, the drive surface 154 is defined by an outward-facing surface of the drive portion 142. The drive surface 154 is configured to be engaged by an inward-facing surface of a drive member of a catheter. The illustrated drive surface 154 has a square shape in a plane that is perpendicular to the longitudinal axis 148. However, other shapes can also be used. Moreover, although the illustrated drive surface 154 is an outward-facing surface, the drive surface 154 could be defined by an inward-facing surface of, for example, a tool cavity.

The drive portion 142 defines a length 156 that is sufficient to permit the drive portion 142 to be engaged by a tool, such as a drive member of a catheter. In some configurations, a length of the drive surface 154 is equal to the length 156 of the drive portion 142.

The suture mount portion 144 is movable relative to one or both of the anchor portion 140 and the drive portion 142. In some configurations, the suture mount portion 144 is movable relative to both the anchor portion 140 and the drive portion 142. In the illustrated arrangement, the suture mount portion 144 is rotatable relative to one or both of the anchor portion 140 and the drive portion 142. Preferably, the suture mount portion 144 is rotatable about the longitudinal axis 148 of the tissue anchor 102.

In some configurations, the suture mount portion 144 comprises a cylindrical body portion 158 having a relatively small length 160 or dimension extending along the longitudinal axis 148. In some configurations, the length 160 is smaller than a diameter 162 or a maximum dimension in a direction perpendicular to the longitudinal axis 148. The body portion 158 includes a cylindrical sidewall 164 that defines a peripheral surface of the body portion. The cylindrical sidewall 164 surrounds and, preferably, extends in a direction parallel to the longitudinal axis 148. The cylindrical sidewall 164 defines a center point or axis 168. Preferably, the center point or axis 168 is offset from the longitudinal axis 148 of the tissue anchor 102.

The suture mount portion 144 comprises a suture mount location 170 configured to connect to, engage or otherwise support a suture, line or other tension member. The suture mount location 170 allows the suture 106 to extend from the tissue anchor 102 in a generally perpendicular direction relative to the longitudinal axis 148. As used herein, the suture 106 extending in a generally perpendicular direction means that the suture 106 is oriented closer to the perpendicular direction than a parallel direction.

In some configurations, the suture mount location 170 is configured to allow the suture mount portion 144 and the tissue anchor 102 to slide on the suture 106. In the illustrated arrangement, the suture mount location 170 comprises a passage 172 that extends through the body portion 158 of the suture mount portion 144 from a first surface 174 to a second surface 176. The first surface 174 is nearer the drive portion 142 and the second surface 176 is nearer the anchor portion 140. In some configurations, the passage 172 extends in a direction generally parallel to the longitudinal axis 148. The passage 172 of the first tissue anchor 102 allows the suture 106 to be tied or otherwise fixedly secured to the first tissue anchor 102. The passage 172 of the second tissue anchor 104 allows the second tissue anchor 104 to slide along the suture 106 so that the tensioned length 132 can be adjusted. As used herein, the term connect when used to describe the interaction between the suture 106 and the suture mount portion 144 can cover both of these situations unless indicated otherwise.

Preferably, the passage 172 is located on an opposite side of the center point or axis 168 from the longitudinal axis 148. Accordingly, a portion of the body portion 158 that includes the passage 172 is oriented in the direction of force acting on the suture 106. The body portion 158 protrudes from the longitudinal axis 148 a greater distance on the side of the passage 172 in comparison to the side opposite the passage 172. In the illustrated arrangement, the suture 106 extends from an end of the passage 172 closest to the anchor portion 140. Such an arrangement advantageously positions the suture 106 close to the tissue surface to inhibit or reduce leaning of the tissue anchor 102 when the suture 106 is tensioned.

In some configurations, the length 151 of the anchor portion 140 is greater than one or both of the length 156 of the drive portion 142 and the length 160 of the suture mount portion 144. In the illustrated arrangement, the length 151 of the anchor portion 140 is greater than both the length 156 of the drive portion 142 and the length 160 of the suture mount portion 144. In some configurations, the length 156 of the drive portion 142 is greater than the length 160 of the suture mount portion 144.

### Suture Lock

The suture lock 110 includes a first portion or base 180. A second portion or cap 182 of the suture lock 110 is movable relative to the base 180 along a longitudinal axis 184 of the suture lock 110. The base 180 and the cap 182 are rotationally fixed relative to one another. A threaded fastener 186 passes through an opening 190 in the cap 182 and engages a threaded cavity 192 of the base 180. Rotation of the threaded fastener 186 in a first direction moves the cap 182 toward the base 180 and rotation of the threaded fastener 186 in a second, opposite direction moves the cap 182 away from the base 180. Accordingly, the suture lock 110 can clamp a lock portion 188 of the suture 106 between the base 180 and the cap 182, release the suture 106 to allow for adjustment of the position of the suture lock 110 relative to the suture 106, and then re-clamp the suture 106.

The base 180 and the cap 182 include cooperating structures that inhibit or prevent relative rotation. The cooperating structures can be one or more flat surfaces or non-circular surfaces relative to the longitudinal axis 184.

In the illustrated arrangement, the base 180 is generally cylindrical in shape. The base 180 includes a protruding portion in the form of a central hub 200 that defines at least one non-circular surface (e.g., a flat surface 202). In the illustrated arrangement, the hub 200 defines a pair of flat surfaces 202 that are spaced from one another on opposite sides of the longitudinal axis 184. The illustrated base 180 is symmetrical about the longitudinal axis 184. Accordingly, the flat surfaces 202 as shown are equidistant from the longitudinal axis 184. As used herein with respect to a structure that inhibits or prevents rotation, a non-circular surface is one in which the surface can cooperate with another surface to inhibit or prevent rotation about the longitudinal 184. Such surfaces can include, for example, flat surfaces or curved surfaces that have a curvature about a center that is not located on the longitudinal axis 184.

The flat surfaces 202 each have at least a component that extends in a direction parallel to the longitudinal axis 184. In the illustrated arrangement, the flat surfaces 202 each are oriented parallel to the longitudinal axis 184. Accordingly, the flat surfaces 202 permit axial movement of the cap 182 relative to the base 180 but inhibit or prevent rotational movement of the cap 182 relative to the base 180.

Each of the flat surfaces 202 is created by a cutout section of a cylindrical work piece that extends only partially through the work piece in a longitudinal direction such that the base 180 also includes at least one flange portion or a base flange 204. In the illustrated arrangement, the base 180 includes a pair of flange portions, which are referred to for convenience hereinafter as flanges 204. Each flange 204 defines a shoulder surface or shoulder 206 adjacent the flat surfaces 202. The shoulders 206 provide a stop surface to limit axial movement of the cap 182 along the longitudinal axis 184. The shoulders 206 also provide a surface against which the suture 106 can be clamped, as is described further below.

The cap 182 is generally cylindrical in shape with a central cut-out defining a space 210 that receives a portion of the base 180. In particular, the space 210 receives the hub 200 of the base 180. The illustrated cap 182 defines an end wall portion 212 and a pair of (e.g., a first and a second) depending side wall portions 214 that cooperate to define the space 210. The end wall portion 212 defines the opening 190 through which the threaded fastener 186 passes. The first and second side wall portions 214 each define a surface 216 that cooperates with one of the flat surfaces 202 to inhibit or prevent relative rotation between the base 180 and the cap 182. The surfaces 216 can be non-circular. In the illustrated arrangement, the surfaces 216 of the side wall portions 214 are flat. Thus, in the illustrated arrangement, both the surfaces 202 of the base 180 and the surfaces 216 are flat. However, other arrangements are possible in which only one of the surfaces 202, 216 are flat or in which neither of the surfaces 202, 216 are flat, but are otherwise configured to cooperate with one another to inhibit or prevent rotation between the base 180 and the cap 182. The illustrated flat surfaces 216 of the cap 182 are in sliding contact with the flat surfaces 202 of the base 180 to permit axial movement and inhibit or prevent rotational movement of the cap 182 relative to the base 180.

The ends of the side wall portions 214 opposite the end wall portion 212 terminate in outwardly or radially-extending flanges 220. The portions of the side wall portions 214 adjacent the flanges 220 define flat surfaces 222. The flat surfaces 222 are parallel to one another in the illustrated arrangement but could be non-parallel in other configurations. The flat surfaces 222 are located radially inward from an outermost extent of the flanges 220 to define a stop surface or shoulder 224. The flat surfaces 222 can be utilized so that a tool (e.g., a catheter) can hold the cap 182 against rotation while the threaded fastener 186 is rotated to move the base 180 and the cap 182 toward or away from one another along the longitudinal axis 184.

As described above, the suture 106 can be captured or clamped between the base 180 and the cap 182. The suture 106 can be captured or clamped between the flange 204 of the base 180 and the corresponding flange 220 of the cap 182. In some configurations, one or both of the base 180 and the cap 182 include a suture retention feature configured to retain the suture 106 to the base 180 and/or cap 182 or at least inhibit or prevent complete separation of the suture 106 from the base 180 and/or cap 182. In the illustrated arrangement, at least one of the flanges 204 of the base 180 includes a suture passage 226 configured to accommodate the suture 106. However, in other arrangements, at least one of the flanges 220 of the cap 182, or both the flange(s) 204 of the base 180 and the flange(s) 220 of the cap 182, can include a suture passage 226.

In the illustrated arrangement, the suture passage 226 extends through the flange 204 from an end surface 230 to the shoulder surface 206. In some configurations, the passage 226 extends in a direction generally parallel to the longitudinal axis 184. As used herein, generally parallel means that the passage 226 is oriented closer to the parallel direction than a perpendicular direction. The passage 226 allows the suture 106 to be retained to the base 180 of the suture lock 110. The passage 226 allows the tissue anchor 110 to slide along the suture 106. The passage 226 retains a portion of the suture 106 between the flange 204 of the base 180 and the flange 220 of the cap 182 so that the suture 106 can be selectively clamped by movement of the cap 182 toward the base 180.

The threaded fastener 186 can be, or can be similar to, a socket head cap bolt. The threaded fastener 186 has a threaded shaft portion or shaft 232 and a head portion or head 234. The head 234 has a larger diameter or cross-sectional size than the shaft 232. The head 234 can define a surface or surfaces configured to engage a tool. In the illustrated arrangement, the head 234 defines a tool cavity 236, such as a hexagon-shaped tool cavity. The shaft 232 passes through the opening 190 of the cap 182 and engages the threaded cavity 192 of the base 180. The head 234 contacts the end wall portion 212 of the cap 182 to retain the cap 182 on the base 180. As described previously, contact between the head 234 and the end wall portion 212 allows the threaded fastener 186 to selectively move the cap 182 toward the base 180 to clamp the suture 106 or to allow the cap 182 to move away from the base 180 to release the suture 106.

### Delivery Catheter(s)

As described previously, the system 100 utilizes one or more catheters to deliver and implant or install the components of the system 100 within the desired anatomy of the patient, such as the mitral valve 114 of the heart in the illustrated application. The catheters can be steerable catheters, as is known in the art. In some implementations, the system 100 includes an anchor delivery catheter 250 configured to deliver one or both of the tissue anchors 102, 104 from outside of the patient to within the heart of the patient. The delivery catheter 250 is configured to implant the tissue anchors 102, 104 within the desired tissue of the patient, such as the mitral valve 114.

The delivery catheter 250 includes an elongate catheter body or tube 252. A handle 254 can be connected to the external end of the tube 252 and can be configured to allow a user to control the delivery catheter 250. A delivery end of the tube 252 that is inserted into the patient includes a tip 256 that is configured to engage the tissue anchors 102, 104. The illustrated tip 256 has a first portion 260 and a second portion 262. The first portion 260 is a stationary portion that is secured to the tube 252 in a rotationally fixed manner. The second portion 262 is a rotatable portion that is rotatable relative to the first portion 260 and, thus, to the tube 252.

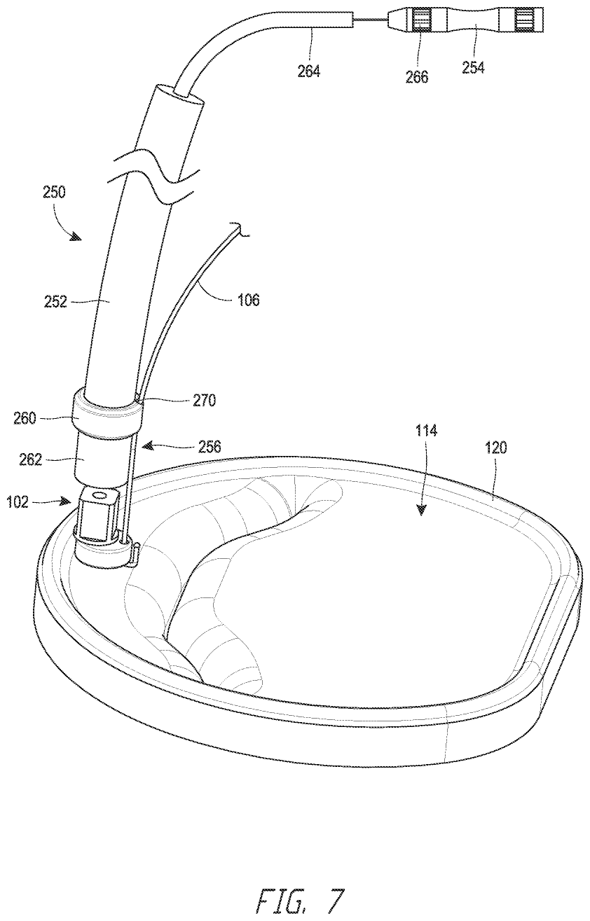

The delivery catheter 250 includes a drive element configured to selectively rotate the second portion 262 of the tip 256. In the illustrated arrangement, the drive element is an elongate drive shaft 264 that extends through the tube 252 from the handle 254 to the second portion 262 of the tip 256. The drive shaft 264 is coupled to the second portion 262 of the tip 256 in a manner such that torque can be transferred from the drive shaft 264 to the second portion 262 of the tip 256. Accordingly, rotation of the drive shaft 264 causes rotation of the second portion 262 of the tip 256. Rotation of the drive shaft 264 can be actuated from the handle 254, such as via a dial or knob 266 or other suitable control member. The handle 254 in FIG. 7 is not shown to scale.

The first portion 260 of the tip 256 can have a diameter or cross-sectional dimension that is larger than the diameter or cross-sectional dimension of the second portion 262 of the tip 256 and/or the tube 252. Preferably, the diameter or cross-sectional dimension of the first portion 260 of the tip 256 is larger than the diameter or cross-sectional dimension of both the second portion 262 of the tip 256 and the tube 252. The first portion 260 of the tip 256 can include a suture passage 270 configured to accommodate the suture 106. The suture passage 270 can extend generally in an axial direction of the delivery catheter 250. Preferably, the delivery catheter 250 is a “rapid exchange” type catheter in which the suture 106 passes through only a small portion of the catheter 250 and is otherwise external of the catheter 250. In the illustrated configuration, the suture 106 passes only through the suture passage 270 of the tip 256 and is completely external of the tube 252.

The second portion 262 of the tip 256 defines an engagement portion configured to engage the tissue anchor 102, 104 and to transfer torque from the second portion 262 of the tip 256 to the tissue anchor 102, 104. In the illustrated arrangement, the second portion 262 of the tip 256 defines a tool cavity 272 configured to receive the drive portion 142 of the tissue anchor 102, 104. The tool cavity 272 and the drive portion 142 each have non-circular cross-sectional shapes that are complementary to one another. In the illustrated arrangement, each of the tool cavity 272 and the drive portion 142 have a square cross-sectional shape. Thus, the drive portion 142 of the tissue anchor 102, 104 can slide into the tool cavity 272 of the second portion 262 of the tip 256. Accordingly, the tissue anchor 102, 104 can be selectively engaged to and disengaged from the tip 256 of the delivery catheter 250. In addition, rotation of the second portion 262 of the tip 256 causes rotation of the drive portion 142 of the tissue anchor 102, 104.

The illustrated system 100 also includes a suture lock delivery catheter 280 configured to deliver and install the suture lock 110. The delivery catheter 280 can be similar to the delivery catheter 250 that delivers the tissue anchors 102, 104. The illustrated delivery catheter 280 includes an elongate catheter body or tube 282. A handle 284 can be connected to the external end of the tube 282 and can be configured to allow a user to control the delivery catheter 280. A delivery end of the tube 282 that is inserted into the patient includes a tip 286 that is configured to engage the suture lock 110. The tip 286 is secured to the tube 282 in a rotationally fixed manner.

The tip 286 can have a diameter or cross-sectional dimension that is larger than the diameter or cross-sectional dimension of the tube 282. The tip 286 can include a suture passage 290 configured to accommodate the suture 106. The suture passage 290 can extend generally in an axial direction of the delivery catheter 280. Preferably, the delivery catheter 280 is a “rapid exchange” type catheter in which the suture 106 passes through only a small portion of the catheter 280 and is otherwise external of the catheter 280. In the illustrated configuration, the suture 106 passes only through the suture passage 290 of the tip 286 and is completely external of the tube 282.

The tip 286 defines an engagement portion configured to engage the suture lock 110. In particular, the tip 286 is configured to hold the cap 182 of the suture lock 110 and inhibit or prevent rotation of the cap 182 so the threaded fastener 186 can be rotated relative to the cap 182 to move the base 180 toward or away from the cap 182. In the illustrated arrangement, the tip 286 defines a cavity 292 configured to receive the cap 182 of the suture lock 110. The cavity 292 includes engagement surfaces 294 that engage the flat surfaces 222 of the cap 182 of the tissue anchor 110. Thus, the cap 182 of the suture lock 110 can slide into the cavity 292 of the tip 286. Accordingly, the cap 182 of the suture lock 110 can be selectively engaged to and disengaged from the tip 286 of the delivery catheter 280. In addition, the tip 286 can hold the cap 182 of the suture lock 110 against rotation.

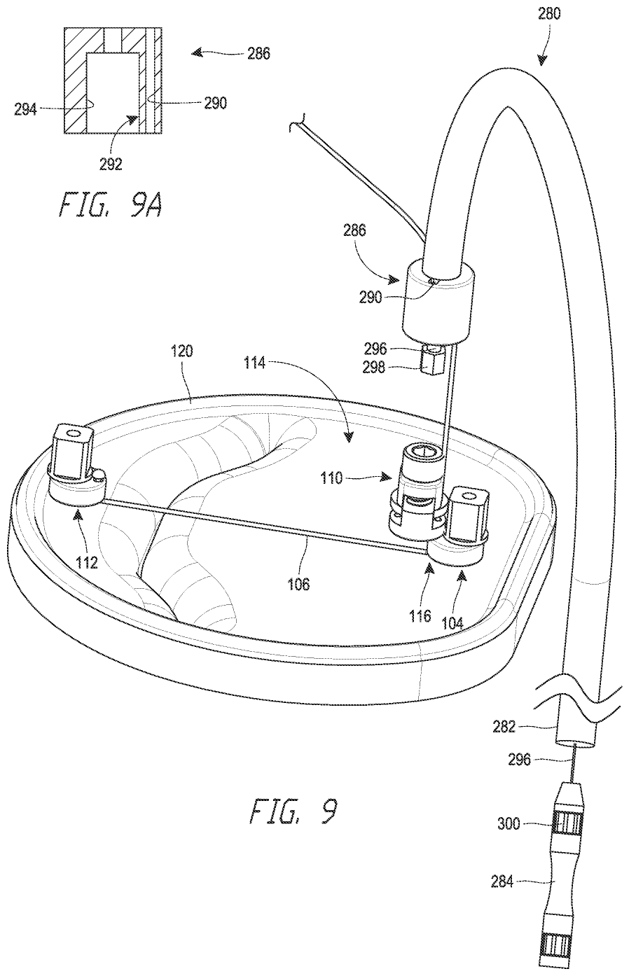

The delivery catheter 250 includes a drive element configured to selectively rotate the threaded fastener 186 of the suture lock 110. In the illustrated arrangement, the drive element is an elongate drive shaft 296 that extends through the tube 282 from the handle 284 to the tip 286. The drive shaft 296 carries a drive element, such as a drive tip or drive tool 298 that is configured to transfer torque from the drive shaft 296 to the threaded fastener 186. In the illustrated arrangement, the drive tool 298 has a shape that is complementary to the tool cavity 236 of the threaded fastener 186. Accordingly, rotation of the drive shaft 296 causes rotation of the threaded fastener 186. Rotation of the drive shaft 296 can be actuated from the handle 284, such as via a dial or knob 300 or other suitable control member. The handle 284 in FIG. 9 is not shown to scale.

### Suture Trimmer

The system 100 can also include a suture trimmer 350 configured to cut off or trim the excess portion of the suture 106. In the illustrated arrangement, the suture trimmer 350 includes an elongate catheter body or tube 352. A handle 354 can be connected to the external end of the tube 352 and can be configured to allow a user to control the suture trimmer 350. A trimming end of the tube 352 that is inserted into the patient includes a tip 356 that is configured to trim the suture 106.

The illustrated tip 356 has a first portion 360 and a second portion 362. The second portion 362 is axially movable relative to the first portion 360. The first portion 360 supports or houses a cutting blade 364. The second portion 362 is configured to receive and retain the suture 106 for cutting by the cutting blade 354. The second portion 362 defines a suture passage 366 configured to accommodate the suture 106. The suture passage 366 extends in a radially or a generally radial direction of the tube 352. That is, the suture passage 366 can extend in a radial direction or a direction that is oblique relative to a longitudinal axis of the tube 352.

The illustrated second portion 362 of the tip 356 also includes a slot 370 configured to receive the cutting blade 364when the second portion 362 is moved axially toward the first portion 360. The slot 370 intersects the suture passage 366. Accordingly, when the suture 106 is located within the suture passage 366, the cutting blade 364 can be moved into the slot 370 to cut the suture 106 by movement of the second portion 362 of the tip 356 toward the first portion 360. In the illustrated arrangement, the slot 370 extends through the end of the second portion 362 of the tip 356. However, in other arrangements, the slot 370 could have a closed end. In other arrangements, the second portion 362 of the tip can be stationary and the blade 364 can be configured to move.

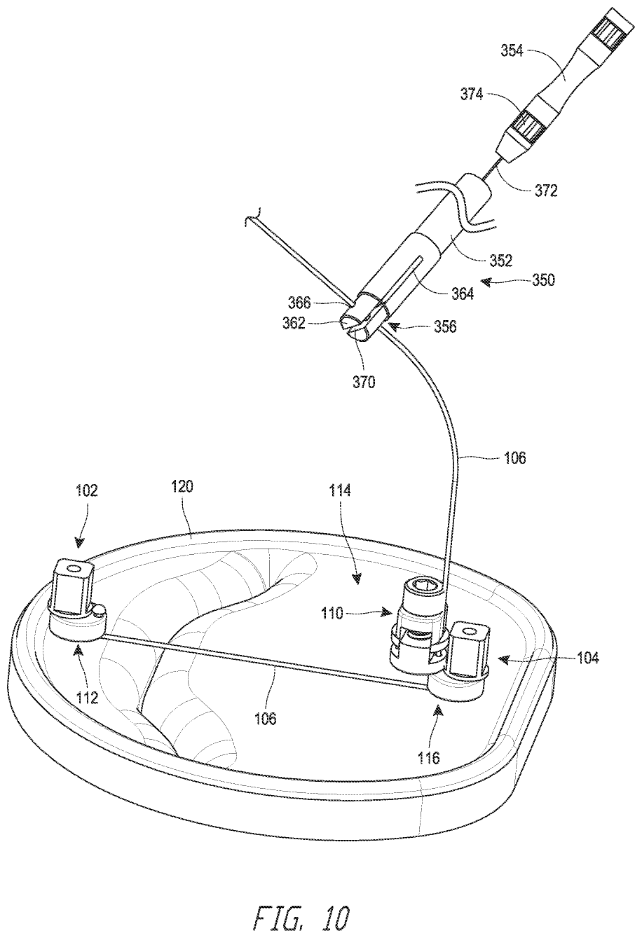

The suture trimmer 350 includes an actuator for moving the second portion 362 toward the first portion 360 for advancing the cutting blade 364 into the slot 370. In the illustrated arrangement, the suture trimmer 350 includes an actuation wire or shaft 372. The actuation wire 372 extends from the handle 354 to the second portion 362 of the tip 356. A user control element, such as a button, knob, dial or lever 374, can be located on the handle 374 and coupled to the actuation wire 372. The control element 374 can apply a pulling force on the actuation wire 372 tending to move the second portion 362 of the tip 356 in an axial direction toward the first portion 360. As a result, the cutting blade 364 is advanced through the slot 370 to cut the suture 106. The handle 354 in FIG. 10 is not shown to scale.

Advantageously, the illustrated arrangement allows the suture 106 to be trimmed at a location close to the suture lock 110. As a result, a relatively short length of excess suture 106 remains within the patient. For example, the excess portion of the suture 106 can be equal to or less than a radius of the tip 356, such as a second portion 362 of the tip 356, if the slot 370 is located in a center of the tube 352 or the tip 356 and the suture passage 366 is oriented in a radial direction of the tube 352 or the tip 356. Accordingly, the second portion 362 of the tip 356 can be configured to have a smaller diameter or cross-sectional dimension than one or both of the first portion 360 of the tip 356 and the tube 352.

### Method

The components of the system 100 can be delivered to the mitral valve 114 of the patient by any suitable method. In some configurations, the components of the system 100 are routed to the left atrium via a transeptal approach, wherein an incision is made in the atrial portion of the septum to allow access from the right atrium, such as via the inferior or superior vena cava. A guide catheter 310 can be routed to the left atrium by any suitable method, such as any transvascular approach as is known in the art. The guide catheter 310 can be configured to receive the delivery catheters 250, 280.

In one method of implantation of the system 100, the suture 106 is attached to the first tissue anchor 102 by any suitable arrangement or method, as indicated at block 400. For example, the suture 106 can be passed through the passage 172 of the suture mount location 170 of the first tissue anchor 102 and tied to itself using a suitable knot. Preferably, the suture 106 extends from the second surface 176 of the anchor portion 140 so that the suture 106 is located adjacent to the tissue of the mitral valve 114.

At block 402, the first tissue anchor 102 can be loaded onto the delivery catheter 250. For example, the suture 106 can be passed through the suture passage 270 of the first portion 260 of the tip 256 of the catheter 250. The suture 106 can be passed through the passage 270 in a direction from the tip 256 toward the tube 252. The drive portion 142 of the first tissue anchor 102 can be inserted into the tool cavity 272 of the tip 256 of the catheter 250.

At block 404, the delivery catheter 250 can be used to deliver the first tissue anchor 102 to the first location 112. For example, the delivery catheter 250 can be passed through the guide catheter 310 to the first location 112, using a suitable guidance technique. During delivery, the suture 106 can be tensioned to help maintain the first tissue anchor 102 in engagement with the tip 256.

At block 406, the first tissue anchor 102 is implanted at the first location 112. For example, the knob 266 can be used to rotate the drive shaft 264, which rotates the second portion 262 of the tip 256 of the catheter 250. Rotation of the second portion 262, in turn, rotates the drive portion 142 and anchor portion 140 of the first tissue anchor 102. Rotation of the anchor portion 140 screws the first tissue anchor 102 into the tissue of the mitral valve 114 at the first location 112. Tension can be kept on the suture 106 to inhibit or prevent rotation of the mount portion 144 of the first tissue anchor 102, which can keep the suture 106 from wrapping around the delivery catheter 250.

At block 408, the delivery catheter 250 is withdrawn from the guide catheter 310 leaving the first tissue anchor 102 in place at the first location 112 of the mitral valve 114. The suture 106 can be removed from the tip 256 of the delivery catheter 250.

At block 410, the second tissue anchor 104 can be loaded onto the delivery catheter 250. For example, the suture 106 can be passed through the suture passage 172 of the second tissue anchor 104. The suture 106 can be passed through the suture passage 172 in a direction from the second surface 176 to the first surface 174 so that the suture 106 is located adjacent to the tissue of the mitral valve 114. The suture 106 can be passed through the suture passage 270 of the first portion 260 of the tip 256 of the catheter 250. The suture 106 can be passed through the passage 270 in a direction from the tip 256 toward the tube 252. The drive portion 142 of the second tissue anchor 104 can be inserted into the tool cavity 272 of the tip 256 of the catheter 250.

At block 412, the delivery catheter 250 can be used to deliver the second tissue anchor 104 to the second location 116. For example, the delivery catheter 250 can be passed through the guide catheter 310 to the second location 116, using a suitable guidance technique. During delivery, the suture 106 can be tensioned to help maintain the second tissue anchor 102 in engagement with the tip 256.

At block 414, the second tissue anchor 104 is implanted at the second location 116. For example, the knob 266 can be used to rotate the drive shaft 264, which rotates the second portion 262 of the tip 256 of the catheter 250. Rotation of the second portion 262, in turn, rotates the drive portion 142 and anchor portion 140 of the second tissue anchor 104. Rotation of the anchor portion 140 screws the second tissue anchor 104 into the tissue of the mitral valve 114 at the second location 116. Tension can be kept on the suture 106 to inhibit or prevent rotation of the mount portion 144 of the second tissue anchor 104, which can keep the suture 106 from wrapping around the delivery catheter 250.

At block 416, the delivery catheter 250 is withdrawn from the guide catheter 310 leaving the second tissue anchor 104 in place at the second location 116 of the mitral valve 114. The suture 106 can be removed from the tip 256 of the delivery catheter 250.

At block 418, the suture lock 110 can be loaded onto the delivery catheter 280. For example, the suture 106 can be passed through the suture passage 226 of the base 180 of the suture lock 110. The suture 106 can be passed through the suture passage 226 in a direction from the end surface 230 to the shoulder surface 206. The suture 106 can be passed through the suture passage 290 of the tip 286 of the delivery catheter 280. The drive tool 298 can be inserted into the tool cavity 236 of the threaded fastener 186. The cap 182 can be inserted into the cavity 292 of the tip 286 of the delivery catheter 280.

At block 420, the suture lock 110 can be delivered to the second location 116 using the delivery catheter 280. For example, the delivery catheter 280 can be passed through the guide catheter 310 to the second location 116, using a suitable guidance technique. During delivery, the suture 106 can be tensioned to help maintain the suture lock 110 in engagement with the tip 286.

At block 422, the tension length 132 of the suture 106 can be adjusted. For example, the end surface 230 of the suture lock 110 can be positioned against the first surface 174 of the suture mount portion 144 of the second tissue anchor 104. The suture 106 can be pulled and the column strength of the delivery catheter 280 can hold the second tissue anchor 104 and suture lock 110 in place. Thus, the suture 106 can be pulled through the respective suture passages 172, 226, 290 of the second tissue anchor 104, the suture lock 110 and the tip 286 of the catheter 280. As a result, the first tissue anchor 102 is pulled toward the second tissue anchor 104 and the tension length 132 is reduced. The suture 106 can also be released and the inherent resiliency of the tissue of the mitral valve 114 can increase the tension length 132.

At block 424, once a desired tension length 132 has been achieved, the suture lock 100 can be locked to secure the fix the suture 106 relative to the suture lock 110. For example, the knob 300 can be used to rotate the drive shaft 296. Rotation of the drive shaft 296 rotates the drive tool 298, which rotates the threaded fastener 186 of the suture lock 110. Rotation of the threaded fastener 186 causes the base 180 and cap 182 of the suture lock 110 to move toward one another thereby clamping the suture 106 between the shoulder surface 206 of the base 180 and the flange 220 of the cap 182.

At block 430, once the suture lock 110 has been locked, but before the delivery catheter 280 has been removed from the suture lock 100, the performance or operation of the mitral valve 114 can be monitored by any suitable imaging process.

At block 426, the delivery catheter 280 can be withdrawn leaving the suture lock 110 in place. For example, the catheter tip 286 and the drive tool 298 can be removed from the cap 182 and the threaded fastener 186, respectively, and the delivery catheter 280 can be withdrawn from the guide catheter 310.

At block 428, the excess portion of the suture 106 can be trimmed. For example, the suture 106 can be passed through the suture passage 366 of the suture trimmer 350. The end of the suture trimmer 350 comprising the tip 356 can be inserted into the guide catheter 310 and advanced to the second location 116. The tip 356 slides along the suture 106 such that an end of the suture 106 remains outside of the patient. The suture 106 can guide the tip 356 of the suture trimmer 350 to the suture lock 110. Once the tip 356 of the suture trimmer 350 is located adjacent the suture lock 110, the control element 374 can be actuated to advance the cutting blade 364 within the slot 370 and cut the suture 106. The external end of the suture 106 can be held to maintain tension in the excess portion of the suture 106 to allow for easier cutting.

As described above, the system 100 is configured such that the tension length 132 can be set, the operation of the mitral valve 114 monitored and, if desired, the tension length 132 changed. This process can be repeated until a desired result is achieved.

Optionally, returning to block 430, once the suture lock 110 has been locked, but before the delivery catheter 280 has been removed from the suture lock 100, the performance or operation of the mitral valve 114 can be monitored by any suitable imaging process.

Optionally, at block 432, if an adjustment is desired, the suture lock 110 can release the suture 106 to allow for adjustment of the tension length 132. For example, the knob 300 can be used to rotate the drive shaft 296 and drive tool 298 in a counter-clockwise direction (or opposite the direction used to clamp the suture 106). Rotation of the drive tool 298 rotates the threaded fastener 186, which allows the cap 182 to move away from the base 180. As a result, the suture 106 is no longer clamped between the cap 182 and the base 180 and is permitted to move relative to the suture lock 110 and second tissue anchor 104.

The tension length 132 can be adjusted as described above with respect to block 422. Once a desired tension length 132 is obtained, the suture 106 can be locked as described with respect to block 424. The method can then return to block 430 to permit further monitoring and, if desired, further adjustment. If the desired operation or performance of the mitral valve 114 has been achieved, the method can move to block 426 and block 428 to release the suture lock 110 and trim the suture 106, as described above.

### Tissue Anchor Cover

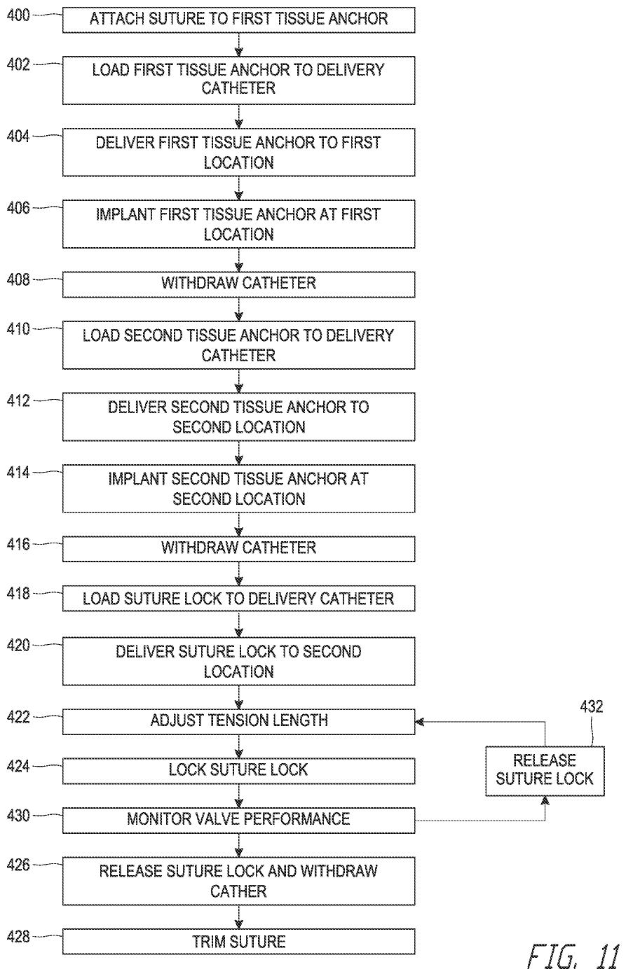

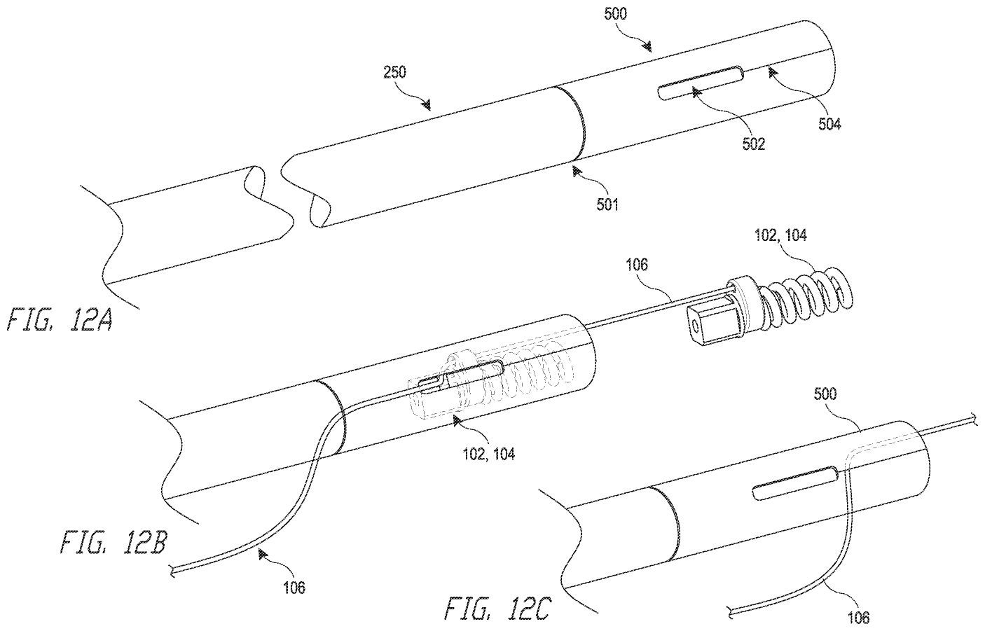

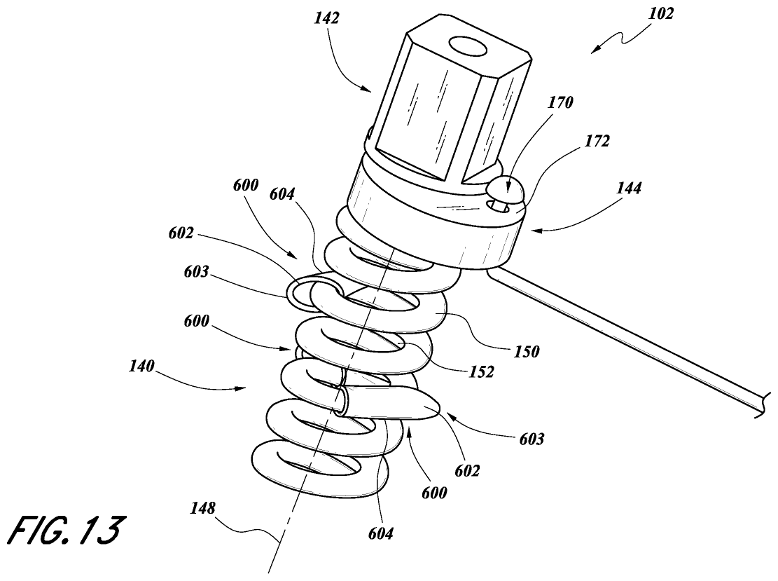

FIGS. 12A-12C illustrate an alternative delivery catheter 250 having a distal tip cover 500 located at the distal end or delivery end of the catheter tube 252. The cover 500 can be configured to accommodate (e.g., cover) the tissue anchor 102, 104 to inhibit or prevent the tissue anchor 102, 104 from damaging tissue (e.g., vasculature or heart tissue) prior to deployment. The cover 500 can be connected, such as bonded or otherwise secured, to or around the tip 256 of the catheter tube 252 at a connection 501. The cover 500 can be generally tubular in form and can define an interior space that receives the tissue anchor 102, 104. The cover 500 can have an open distal end through which the tissue anchor 102, 104 can be deployed.

The cover 500 includes a through hole in the form of a slot 502 that passes in a radial direction through a sidewall of the cover 500. The slot 502 accommodates the suture 106 such that the suture 106 can pass from external of the cover 500 to internal of the cover 500. As described above, the suture 106 engages the tissue anchor 102, 104 that is initially located within the cover 500 prior to deployment. Thus, the suture 106 is located outside of the catheter tube 252 and passes through the slot 502 to engage the tissue anchor 102, 104, as illustrated in FIG. 12B.

The cover 500 also includes a slit 504 that extends lengthwise from the slot 502 to the distal or free end of the cover 500. The slit 504 passes entirely through the sidewall of the cover 500. As a result, the suture 106 can pass from the slot 502 through the slit 504 as the tissue anchor 102, 104 is deployed from the cover 500 thereby allowing the suture 106 can separate or disengage from the cover 500 and the catheter 250. FIG. 12C illustrates the suture 106 passing through the slit 504.

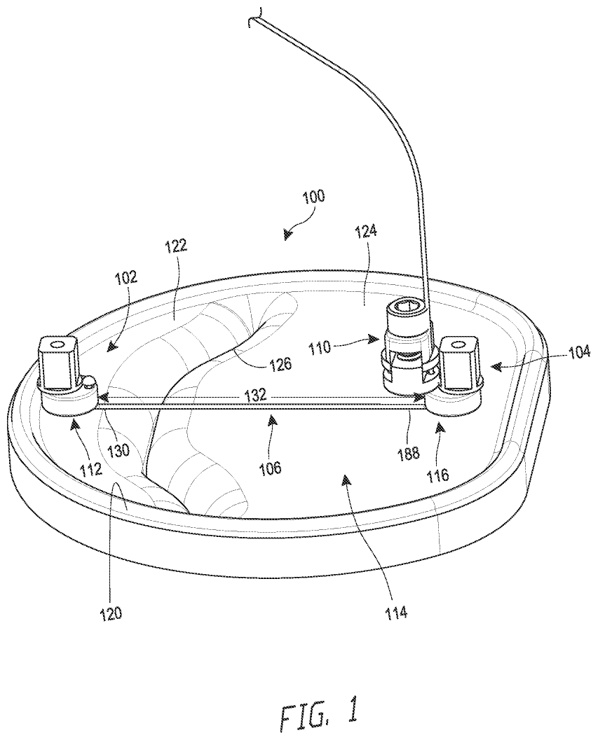

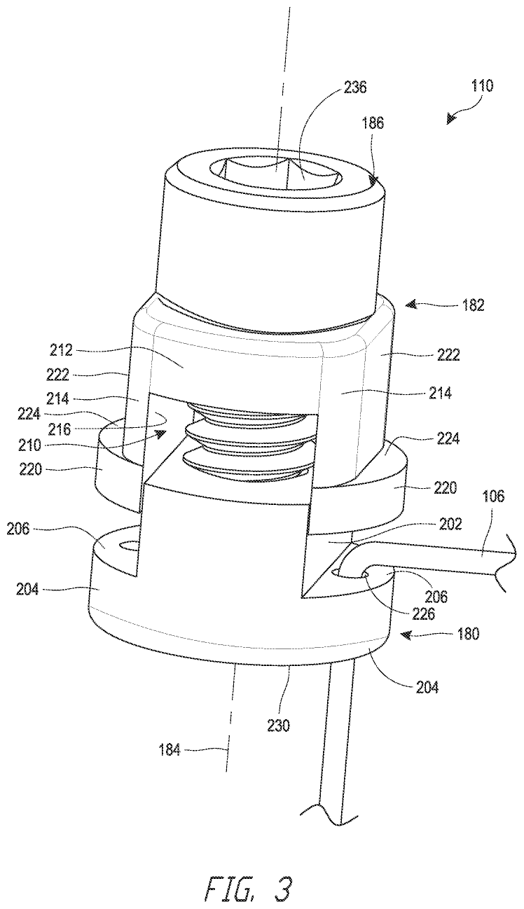

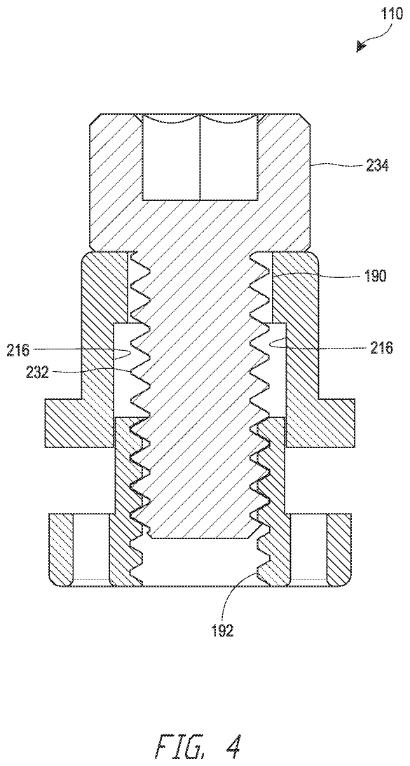

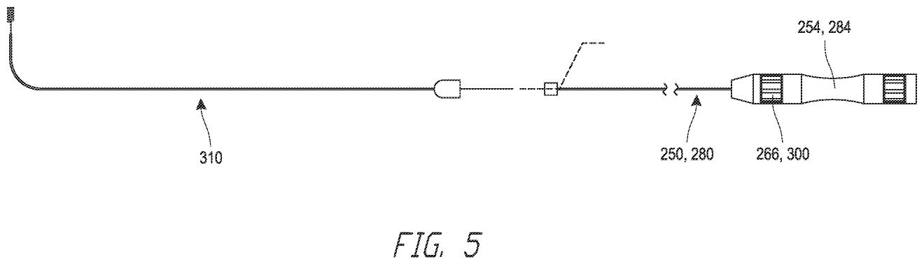

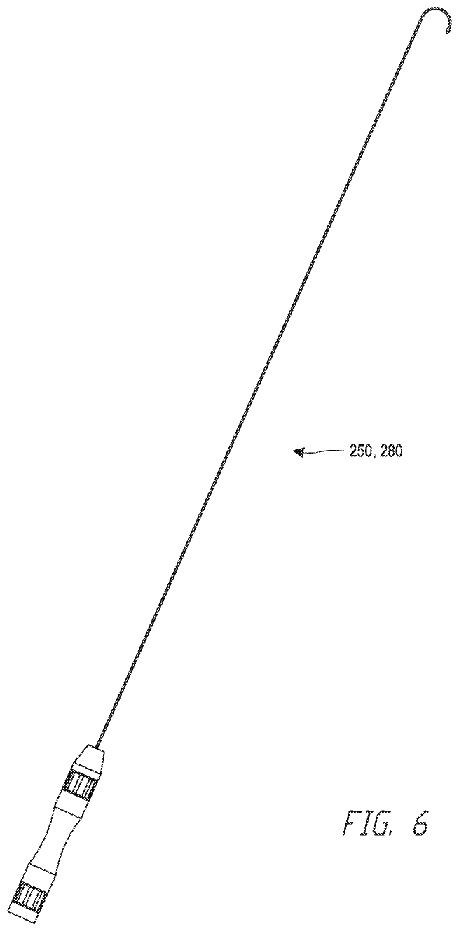

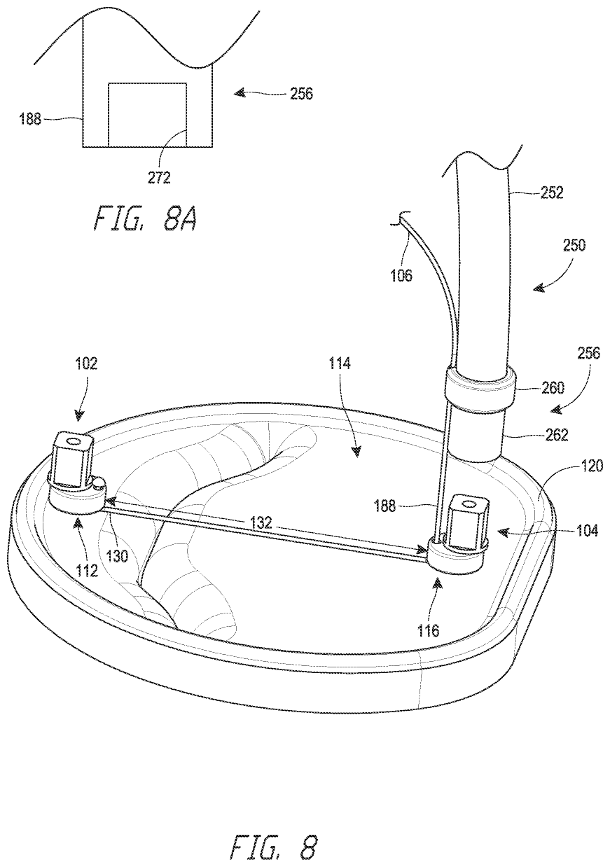

Such an arrangement advantageously provided protection to the tissue anchor 102, 104 and protects other tissues from the sharp end of the tissue anchor 102, 104 until deployment. The cover 500 also allows for simple disengagement of the suture 106 from the cover 500. The cover 500 can be constructed from any suitable material or combination of materials. For example, the cover 500 can be constructed from a suitable medical grade polymer material or materials. The cover 500 can be implemented with the system 100 or components of the system 100 described with respect to FIGS. 1-11, such as the delivery catheter 250 of FIGS. 1-11. Alternatively, the catheter 250 of FIGS. 12A-12C can be utilized with the system 100 of FIGS. 1-11.

Tissue Anchor with Barbs

FIG. 13 illustrates an alternative tissue anchor 102 that provides for improved retention within tissue relative to the tissue anchors 102, 104 described above. The tissue anchor 102 of FIG. 13 is similar to the prior tissue anchors 102, 104 and, therefore, the same reference numbers are used to refer to the same or corresponding components or features. The alternative tissue anchor 102 is described in the context of the differences relative to the previously described tissue anchors 102, 104. Any components or features of the alternative tissue anchor 102 not described in detail can be assumed to be the same as or similar to the component or feature of the tissue anchors 102, 104, or can be of another suitable arrangement. Moreover, the tissue anchor 102 of FIG. 13 can replace either one or both of the tissue anchors 102, 104 in the systems and methods described above.

The illustrated tissue anchor 102 includes an anchor portion 140, a drive portion 142 and a suture mount portion 144 arranged along a longitudinal axis 148 of the tissue anchor 102. In the illustrated configuration, the suture mount portion 144 is located between the anchor portion 140 and the drive portion 142 along the longitudinal axis 148.

The anchor portion 140 is configured to be implanted into soft tissue, such as heart tissue, as described above. The illustrated anchor portion 140 comprises a helical member 150 that is implanted by rotation (e.g., clockwise rotation) about the longitudinal axis 148. The helical member 150 comprises an elongate member having a circular cross-section, which is wound about the longitudinal axis 148 to define an elongate hollow space 152 extending along the longitudinal axis 148.

The anchor portion 140 of the tissue anchor 102 of FIG. 13 includes at least one barb 600 or other similar projection that is configured to impede rotation of the anchor portion 140 in a direction tending to remove the tissue anchor 102 from tissue (e.g., counterclockwise rotation). Preferably, the anchor portion 140 includes a plurality of barbs 600 spaced from one another along a length of the helical member 150 and/or along the longitudinal axis 148. In the illustrated arrangement, the anchor portion 140 includes three barbs 600. However, other numbers of barbs 600 could also be used, such as one, two, four, five, six or more barbs 600, for example and without limitation. The number of barbs 600 can be selected to provide a desired amount of resistance to removal of the tissue anchor 102 for the intended use.

Each barb 600 is configured to permit implantation of the tissue anchor 102 with a first level of resistance and to inhibit removal of the tissue anchor 102 with a second level of resistance that is greater than the first level of resistance. For example, each barb 600 can be configured to allow rotation in an implantation direction (e.g., clockwise rotation) to permit implantation of the tissue anchor 102 and to resist rotation in a removal direction (e.g., counterclockwise rotation). In some configurations, each barb 600 includes an end 602 configured to engage tissue to resist rotation of the anchor portion 140 of the tissue anchor 102 in the removal direction. In some configurations, the end 602 is an angled end that is oriented at an angle relative to the tangential direction of the location on the helical member 150 at which the barb 600 is provided. The angled end 602 is oriented with the tip 603 located radially outward on the barb 600. With such an arrangement, the barb 600 permits rotation in the implantation direction (e.g., clockwise rotation) and resists rotation in the opposite removal direction (e.g., counterclockwise rotation).

In some configurations, each barb 600 is constructed of or comprises a tubular element or tube 604. The tube 604 can define an interior passage that is equal to or somewhat larger in diameter and/or cross-sectional area than the diameter and/or cross-sectional area of the helical member 150. The tube 604 can be formed separately from the helical member 150 an assembled thereto. Preferably, the tube 604 is configured to be movable along the helical member 150 so that the tube 604 can be assembled onto the helical member 150. For example, the tube 604 can be pushed onto the terminal end of the helical member 150 and advanced along the helical member towards the drive portion 142 and suture mount portion 144 of the tissue anchor 102. The tube 604 can be secured at a desired location along the helical member 150 by any suitable arrangement or method. For example, the tube 604 can have an interference fit with the helical member 150 and/or can be secured by soldering, welding or adhesives, for example and without limitation.

In some configurations, the tube 604 may be straight or linear. The angled end 602 can define an angle relative to a longitudinal axis 606 of the tube 604. In some configurations, the tube 604 may be curved along its length. In such configurations, the angle of the angled end 602 can be measured relative to an axis that passes through the geometric center of the cross-section of the tube 604 and is oriented perpendicular to the cross-sectional plane. In some configurations the angle can be between 20-70 degrees, 30-60 degrees, 40-50 degrees, or about 45 degrees.

Although the illustrate barbs 600 are created by member that is formed separately from the helical member 150, other suitable methods or structures for creating the barb(s) 600 can also be used. For example, the barb(s) 600 can be formed in a unitary manner along with the helical member 150. In some configurations, the helical member 150 can be formed with the barb(s) 600 in place, such as by a forming or three-dimensional printing process. In other configurations, a workpiece used to create the helical member 150 could be notched or cut such that the barb(s) 600 are formed when the workpiece is wound to create the helical shape of the helical member 150. Alternatively, the barb(s) 600 could be created by notching or cutting of the helical member 150 after it is provided in the helical form. Furthermore, although the illustrated barbs 600 are integral with and rotation along with the helical member 150, in other configurations the barb(s) 600 or other anti-rotation features could be deployed and/or engaged separately once the tissue anchor 102 has been implanted by a suitable actuation arrangement (e.g., a push rod).

As in the tissue anchors 102, 104 described above, the drive portion 142 of the tissue anchor 102 of FIG. 13 is configured to be engaged by a catheter or other implantation tool to allow for implantation of the tissue anchor 102. The drive portion 142 is fixed for rotation with the anchor portion 140 such that rotation of the drive portion 142 results in rotation of the anchor portion 140.

As in the tissue anchors 102, 104 described above, the suture mount portion 144 of the tissue anchor 102 of FIG. 13 is rotatable relative to one or both of the anchor portion 140 and the drive portion 142. Preferably, the suture mount portion 144 is rotatable about the longitudinal axis 148 of the tissue anchor 102. The suture mount portion 144 comprises a suture mount location 170 configured to connect to, engage or otherwise support a suture, line or other tension member. The suture mount location 170 allows the suture 106 to extend from the tissue anchor 102 in a generally perpendicular direction relative to the longitudinal axis 148.

In the illustrated arrangement, the suture mount location 170 comprises a passage 172 that extends through the body portion 158 of the suture mount portion 144 from a first surface 174 nearer the drive portion 142 to a second surface 176 nearer the anchor portion 140. In some configurations, the passage 172 extends in a direction generally parallel to the longitudinal axis 148. The passage 172 of the tissue anchor 102 allows the suture 106 to be tied or otherwise fixedly secured to the first tissue anchor 102 or allows the tissue anchor 102 to slide along the suture 106 so that the tensioned length 132 (FIG. 1) can be adjusted, as described above. Accordingly, the tissue anchor 102 of FIG. 13 can perform the function of either one of the previously described tissue anchors 102, 104.

The tissue anchor 102 of FIG. 13 provides increased resistance to removal from tissue via one or both of straight pull out or backing out by rotation. Accordingly, under at least some conditions, it is preferably for one or both of the tissue anchors 102, 104 described previously to be replaced with the tissue anchor of FIG. 13 in any of the systems or methods described herein to provide increased resistance to removal from the tissue into which the tissue anchor 102 is implanted. Therefore, any tissue anchor 102, 104 described herein can be replaced by the tissue anchor 102 of FIG. 13 or otherwise modified to include similar barbs or other projections or features that increase retention.

### Suture Lock Retention

In some situations, it may be desirable to retain the suture lock 110 relative to one of the tissue anchors 102, 104. For example, in the event that the suture 106 is severed, it may be desirable to prevent the suture lock 110 from being unrestrained or free floating within the heart or other organ or anatomical location. In some cases, further interventions may be desirable in the place of the remodeling system 100. Such interventions may include additional valve repair or valve replacement. The implementation of these further interventions may necessitate cutting of the suture to reduce or remove the influence of the remodeling system 100. In the remodeling system 100 illustrated in FIG. 1, the suture lock 110 is not permanently coupled to the associated tissue anchor 104, but is held in position against tissue anchor 104 by the tension of the suture 106. Once the suture 106 is cut, the suture lock 110 is no longer held in place relative to the tissue anchor 104 and is capable of moving within the patient's anatomy. In order to avoid this situation, certain embodiments of the remodeling system 100 may be configured to retain the suture lock 110 relative to another component of the system 100, which preferably is an implanted component (e.g., one of the tissue anchors 102, 104) that is secured within the patient's anatomy. The suture lock 110 may be coupled directly or indirectly relative to an associated component (e.g., tissue anchor 102, 104).

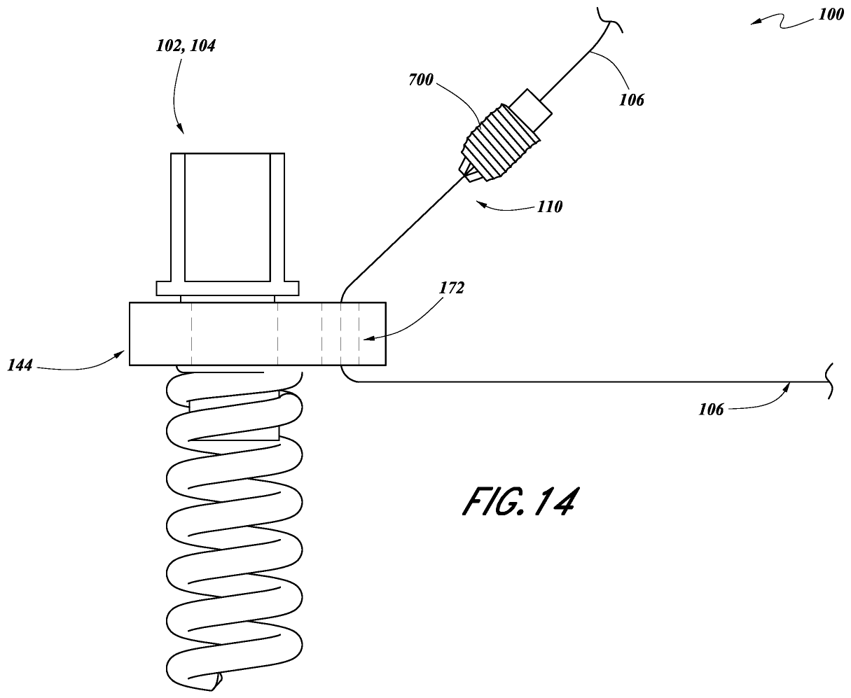

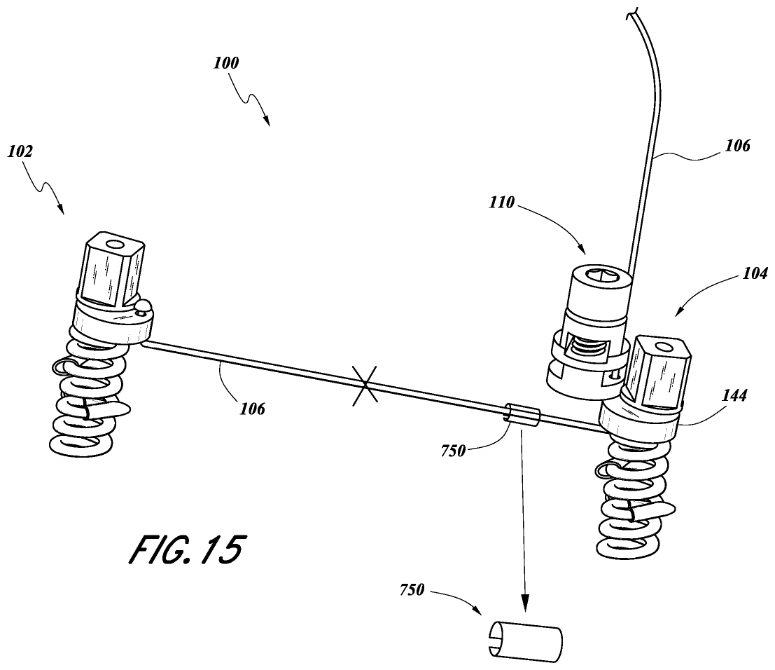

As illustrated in FIG. 14, a suture lock 110 may be configured to be secured to the tissue anchor 102, 104. In particular, the suture lock 110 may be directly secured to the tissue anchor 102, 104. In the illustrated arrangement, the suture lock 110 is configured to be directly secured to the suture mount portion 144 of the tissue anchor 102, 104. For example, the suture lock 110 may be a clamp, such as a chuck or collet, that surrounds the suture 106 and is configured to apply a clamping force to the suture 106 when the suture lock 110 is secured to the tissue anchor 102, 104. However, the clamping force and the coupling to the tissue anchor 102, 104 could also be accomplished separately.

In the illustrated arrangement, the suture lock 110 includes an outer threaded surface 700 that is configured to engage with an inner threaded surface (not shown) of the passage 172 of the tissue anchor 102, 104. The outer threaded surface 700, the inner threaded surface, or both may be tapered or include a tapered portion, or another suitable arrangement may be provided, such that the suture lock 110 clamps the suture 106 as the suture lock 110 is advanced into the passage 172 to both couple the suture 106 to the suture lock 110 and couple the suture lock 110 to the tissue anchor 102, 104. Advantageously, with such an arrangement, if the suture 106 is severed, the suture lock 110 remains secured to the tissue anchor 102, 104. Because the tissue anchor 102, 104 is securely implanted, the suture lock 110 also remains secured in place within the patient's anatomy.

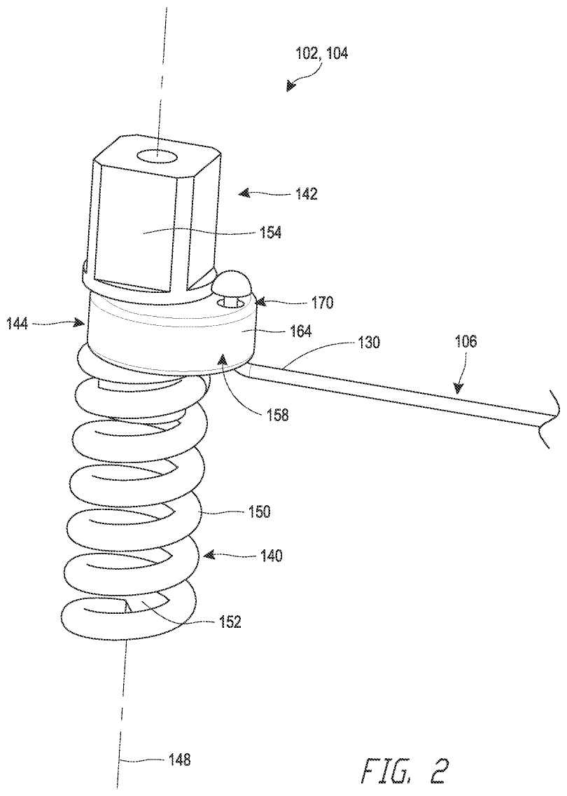

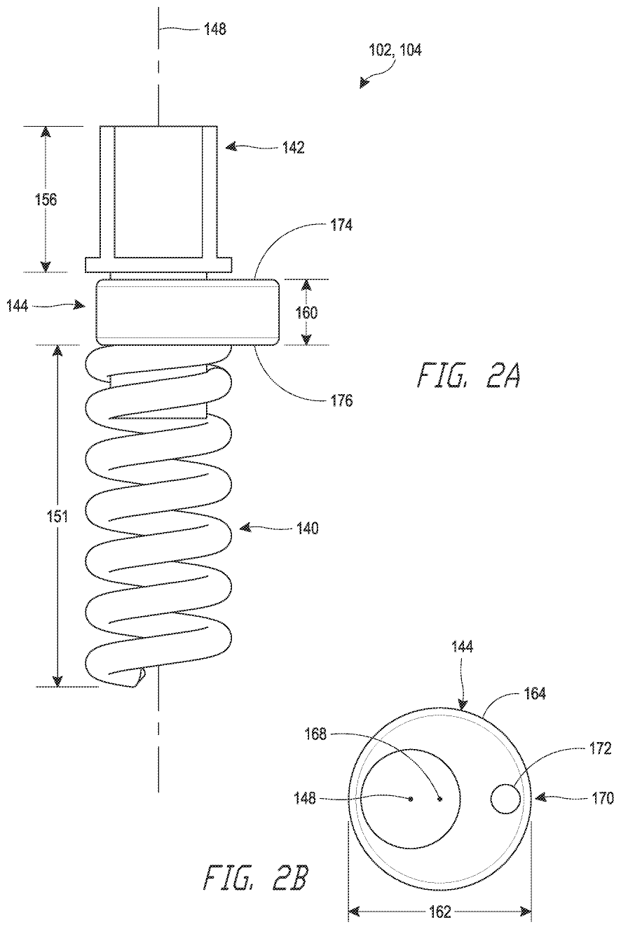

FIG. 15 illustrated another retention arrangement to secure the suture lock 110. In particular, the arrangement of FIG. 15 is configured to retain the suture lock 110 in proximity to the associated tissue anchor 104 using a portion of the suture 106. In the illustrated arrangement, a stop, block or blocking element 750, such as a metal band or ferrule, is secured to the suture 106 between the tissue anchors 102, 104 or on an opposite side of the suture mount portion 144 of the associated tissue anchor 104 from the suture lock 110. The blocking element 750 is configured to be unable to pass through the passage 172 (see, FIGS. 2B and 14) of the tissue anchor 104. As a result, once cut, a portion of the suture 106 that passes through the passage 172 is secured to the tissue anchor 104 by the blocking element 750 on one end and the suture lock 110 on the other end, each being unable to pass through the passage 172.

The blocking element 750 can be any suitable structure that can be secured to the suture and unable to pass through the passage 172. In the illustrated arrangement, the blocking element 750 is a metal band, which is placed onto the suture 106 and can be crimped or otherwise secured in place at a particular location on the suture 106. Preferably, the metal band 750 is threaded onto the suture 106 before the second tissue anchor 104 and suture lock 110 are threaded onto the suture 106 in the delivery method described above. However, it may also be possible to attach the metal band 750 after delivery of both tissue anchors 102, 104, including after delivery of the suture lock 110. Preferably, the metal band 750 is crimped after it has been moved to the implantation site. The metal band 750 may be crimped after the second tissue anchor 104 is delivered or implanted. In some cases, the metal band 750 may be crimped after the entire remodeling system 100 is implanted and adjusted.

The position of the metal band 750 on the suture 106 can influence the degree of permitted movement of the suture lock 110. That is, the length of the suture 106 between the suture lock 110 and the metal band 750 influences or defines the degree of permitted movement of the suture lock 110. Thus, it may be desirable for the metal band 750 to be as close as possible or practical to the tissue anchor 104 associated with the suture lock 110.

As indicated above, the blocking element 750 can be or comprise suitable structures other than a metal band. For example, the blocking element 750 can be any type of clamp having a body portion large enough to prevent the body portion from passing through the passage 172 of the tissue anchor. Other suitable blocking elements 750 can include a retention portion or function that allows the blocking element 750 to be secured in place on the suture 160 and a blocking portion or function that prevents the blocking element 750 from passing through the passage 172 of the tissue anchor 104.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

The invention may also be said broadly to consist in the parts, elements and features referred to or indicated in the specification of the application, individually or collectively, in any or all combinations of two or more of said parts, elements or features.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

What is claimed is:

1. A system for implanting a tissue anchor in heart tissue of a patient, comprising:
a delivery catheter comprising an anchor delivery tip, the tip comprising a stationary portion and a rotatable portion, the stationary portion comprising a suture passage having a first end and a second end, the rotatable portion comprising a drive portion;
a tissue anchor comprising an anchor portion, a drive portion and a suture mount portion, wherein the anchor portion engages the heart tissue and is implanted by rotation about a longitudinal axis of the tissue anchor, wherein the anchor portion of the tissue anchor comprises one or more barbs, wherein the drive portion is rotatably fixed with respect to the anchor portion and is configured to removably engage with the drive portion of the catheter, wherein the suture mount portion is rotatable relative to the anchor portion and the drive portion;
a suture secured to the suture mount portion;
wherein the tissue anchor is configured to be engaged with the delivery catheter with the drive portion of the tissue anchor engaged with the drive portion of the delivery catheter;
wherein the suture extends through the suture passage of the tip of the delivery catheter such that the suture can be tensioned to restrain the suture mount portion of the tissue anchor from rotating as the rotatable portion of the tip of the delivery catheter is rotated to rotate the drive portion and the anchor portion of the tissue anchor to thereby implant the tissue anchor into the heart tissue.

2. The system of claim 1, wherein the suture passage of the stationary portion is located radially outward of the rotatable portion.

3. The system of claim 1, wherein the suture mount portion is located between the drive portion and the anchor portion.

4. The system of claim 1, wherein the delivery catheter comprises a distal tip cover configured to surround the tissue anchor prior to deployment.

5. The system of claim 4, wherein the distal tip cover comprises a slot through which the suture passes from exterior the distal tip cover to interior the distal tip cover such that the suture can be secured to the suture mount portion.

6. The system of claim 5, wherein the distal tip cover comprises a slit that extends from the slot to a distal end of the distal tip cover, wherein the slit is configured such that the suture can move from the slot, pass through the slit, and be separated from the distal tip cover when the tissue anchor is deployed from the delivery catheter.

7. The system of claim 1, wherein each of the barbs comprises a tubular element having an angled end with a tip of the angled end located radially outward.

8. A method of remodeling a mitral valve, comprising:
implanting, using at least one catheter, a first tissue anchor at a first location at or near an annulus of a mitral valve of a patient;
implanting, using the at least one catheter, a second tissue anchor at a second location at or near the annulus of the mitral valve of the patient across from the first location;
extending a suture between the first tissue anchor and the second tissue anchor and using the suture to move the first tissue anchor and the second tissue anchor toward one another;
fixing a tension length of the suture between the first tissue anchor and the second tissue anchor using a suture lock that is lockable using the at least one catheter;
observing the function of the mitral valve and, if desired, unlocking the suture lock, increasing or decreasing the tension length of the suture, and relocking the suture lock;
permanently attaching the suture lock directly or indirectly to one of the first and second tissue anchors.

9. A tissue anchor, comprising:
an anchor portion comprising a helical thread configured to be implanted into bodily tissue by rotation in a first direction about a longitudinal axis of the tissue anchor;
one or more barbs configured to permit rotation of the tissue anchor in the first direction and inhibit rotation of the tissue anchor in a second direction opposite the first direction;
a drive portion that is rotatably fixed with respect to the anchor portion, the drive portion configured to removably engage with a drive member of a catheter such that rotation of the drive member rotates the drive portion and the anchor portion of the tissue anchor;
a suture mount portion configured to connect to a suture at a suture mount location.

10. The tissue anchor of claim 9, wherein the suture mount portion is rotatable relative to the anchor portion and the drive portion.

11. The tissue anchor of claim 10, wherein the suture mount portion is configured to rotate to align the suture mount location with a direction of force of the suture.

12. The tissue anchor of claim 11, wherein the suture mount portion is located between the drive portion and the anchor portion along the longitudinal axis.

13. The tissue anchor of claim 9, wherein the one or more barbs comprises a plurality of barbs spaced from one another along a length of the helical thread.

14. The tissue anchor of claim 9, wherein each of the one or more barbs comprises an angled end having a tip that is located radially outward on the barb.

15. The tissue anchor of claim 9, where each of the one or more barbs is or comprises a tubular element secured to the helical thread.
